(19) United States
(12) Patent Application Publication
(10) Pub. No.: US 2003/0225339 A1
(43) Pub. Date: Dec. 4, 2003

(54) Methods for inducing temporary changes in ventilation for estimation of hemodynamic performance

(75) Inventors: Joseph A. Orr, Park City, UT (US); Kai Kuck, Hamburg (DE); Lara Brewer, Bountiful, UT (US); Michael B. Jaffe, Cheshire, CT (US)

Correspondence Address: MICHAEL W. HAAS, INTELLECTUAL PROPERTY COUNSEL, RESPIRONICS, INC., 1010 MURRY RIDGE LANE, MURRYSVILLE, PA, 15668, US

(73) Assignee: Respironics Novametrix

(21) Appl. No.: 10/424,656

(22) Filed: Apr. 28, 2003

Related U.S. Application Data
(60) Provisional application No. 60/380,094, filed on May 6, 2002.

(51) Int. Cl. A61B 5/08

(52) U.S. Cl. 600/532

(57) ABSTRACT

Methods for inducing changes in the effective ventilation of an individual by varying one or more respiratory parameters to derive measurements which may be utilized to calculate measures of hemodynamic performance, e.g., pulmonary capillary blood flow and cardiac output, are provided. The methods of the present invention are used to attain measurements under the wide array of ventilatory modes available with modem ventilation machines to calculate indicators of hemodynamic performance using a differential form of the carbon dioxide Fick equation.

### CROSS-REFERENCE TO RELATED APPLICATIONS

This applications claims priority under 35 U.S.C. §119(e) from U.S. Provisional Patent Application No. 60/380,094 filed May 6, 2002.

### BACKGROUND OF THE INVENTION

1. Field of the Invention

The present invention relates generally to methods for noninvasively determining the pulmonary capillary blood flow (“PCBF”) or cardiac output (“CO”) of an individual and, more specifically, to methods for inducing temporary changes in the ventilation of an individual to facilitate such noninvasive measurements. In particular, the present invention relates to methods for inducing changes in the effective ventilation of an individual by manipulating one or more respiratory control parameters, as well as to so-called differential Fick techniques for calculating PCBF or CO measurements based on such changes.

2. Background of Related Art

A. Rebreathing

Cardiac output and pulmonary capillary blood flow are commonly measured indicators of hemodynamic performance, which may be employed in the diagnosis and monitoring of individuals having, or suspected of having, cardiac and/or pulmonary dysfunction. Pulmonary capillary blood flow is the blood flow through the lungs of an individual that participates in gas exchange, i.e., a measure of the effectiveness of pulmonary function. Cardiac output is the sum of pulmonary capillary blood flow and the blood flow that does not participate in gas exchange, which is typically referred to as intrapulmonary shunt flow or venous admixture. In simple terms, CO is a measure of the effectiveness of cardiac function. In most instances, intrapulmonary shunt flow is negligible and, thus, CO and PCBF typically are assumed to be equal.

Cardiac output (or pulmonary capillary blood flow) has traditionally been measured by utilizing the basic physiological principle known as the Fick principle. The Fick principle states that the rate of uptake of a substance by the blood or release of a substance from the blood at the lung, i.e., at the alveoli, is equal to the amount of the substance entering the stream of flow divided by the content difference of the substance at each side of the lung (i.e., upstream and downstream from the alveoli and the pulmonary capillaries). Thus, according to the Fick principle, and using oxygen as the measuring substance, as traditionally has been done, oxygen consumption through the lungs and the oxygen content on either side of the lungs is measured and applied to the following formula:

Q=VO₂/(CVO₂−CaO₂), (1)

where VO₂ represents the oxygen consumption, i.e., the amount of oxygen entering the stream of blood flow over a given period of time, CVO₂ represents the mixed venous oxygen content, i.e., oxygen content on one side of the lungs, or upstream from the alveoli and pulmonary capillaries, and CaO₂ represents the arterial oxygen content, i.e., oxygen content on the other side of the lungs, or downstream from the alveoli and pulmonary capillaries.

Use of equation (1) and, in particular, use of oxygen as the measuring substance, presents a number of drawbacks. Specifically, conventional methods for directly measuring mixed venous oxygen content require cardiac catheterization. As is apparent, such an invasive procedure creates the possibility of harming the individual in both the insertion and the positional maintenance of the catheters.

Thus, safer, non-invasive techniques for determining pulmonary capillary blood flow and cardiac output based upon the Fick principle have been developed. One variation is the so-called carbon dioxide Fick technique, in which carbon dioxide is used as the measuring substance and applied to the Fick principle resulting in the following equation:

Q=VCO₂/(CVCO₂−CaCO₂), (2)

where Q represents blood flow, e.g., pulmonary capillary blood flow or cardiac output, VCO₂ represents carbon dioxide elimination, i.e., the amount of carbon dioxide released from the stream of blood flow over a given period of time, CVCO₂ represents the carbon dioxide content of the venous blood of the monitored individual, i.e., CO₂ content on the upstream side of the lungs, and CaCO₂ represents the carbon dioxide content of the arterial blood of the monitored individual, i.e., CO₂ content on the downstream side of the lungs.

Carbon dioxide elimination (VCO₂) is typically measured as the difference between the amount of carbon dioxide inhaled and the amount of carbon dioxide exhaled, with the amount of carbon dioxide exhaled usually being greater than that inhaled. The carbon dioxide elimination of an individual may be noninvasively measured as the difference, per breath, between the volume of carbon dioxide inhaled during inspiration and the volume of carbon dioxide exhaled during expiration. Carbon dioxide elimination is typically calculated using the following, or an equivalent, equation:

$${VCO}_{2} = {\int }_{breath} V \times {f}_{CO2} ⅆ t \qquad ( 3 )$$

where V is the measured respiratory flow and f_{CO2} is the substantially simultaneously detected carbon dioxide signal, or fraction of the respiratory gases that comprises carbon dioxide, i.e., the “carbon dioxide fraction”.

The carbon dioxide content of the venous blood (CvCO₂) may be estimated, or the need to know the value thereof obviated, as more fully described below. A determination of the CaCO₂ of an individual, on the other hand, is typically based upon the measured partial pressure of end-tidal carbon dioxide (PetCO₂ or etCO₂) of the individual, i.e., the partial pressure of carbon dioxide at a predetermined end portion of a breath by the individual. Partial pressure of end-tidal CO₂, after correcting for any deadspace in the individual's airway or in a respiratory conduit, e.g., a breathing circuit, nasal canula, etc., is typically assumed to be approximately equal to the partial pressure of carbon dioxide in the alveoli (PACO₂) of the individual's lungs or, if there is no intrapulmonary shunt, the partial pressure of carbon dioxide in the arterial blood of the individual (PaCO₂). Using a standard carbon dioxide dissociation curve, either the PetCO₂ measurement or the PaCO₂ calculation may be used to determine CaCO₂.

Typically, a differential form of the carbon dioxide Fick equation is used to noninvasively determine the pulmonary capillary blood flow or cardiac output of an individual. Differential Fick techniques for determining the pulmonary capillary blood flow or cardiac output of an individual are based on the fundamental premise that the pulmonary capillary blood flow or cardiac output of an individual can be estimated based upon the changes of other, measurable parameters when a change in the effective ventilation (i.e., the total ventilation less the wasted ventilation due to deadspace associated with the apparatus, the individual, or a combination thereof) occurs.

When a differential form of the Fick equation is used, the pulmonary capillary blood flow or cardiac output of an individual may be determined on the basis of differences in each of VCO₂, CaCO₂, and CvCO₂ during two different states of ventilation, such as “normal” respiration and while a change in the effective ventilation of the individual is being induced. The following is an example of a differential Fick equation:

$${Q}_{pcbfBD} = \frac{{VCO}_{2 B} - {VCO}_{2 D}}{( {CvCO}_{2 B} - {CvCO}_{2 B} ) - ( {CaCO}_{2 B} - {CaCO}_{2 B} )} , \qquad ( 4 )$$

where VCO_{2 B} and VCO_{2 D} represent the carbon dioxide elimination of the individual during “normal” breathing, and while a change in effective ventilation is being induced, respectively, CvCO_{2 B} and CvCO_{2 D} represent the contents of CO₂ of the venous blood of the individual during the same periods, and CaCO_{2B} and CaCO_{2D} represent the content of CO₂ in the arterial blood of the individual during “normal” breathing and when the effective ventilation of the individual is changed, respectively.

Alternative methods for noninvasively determining cardiac output, pulmonary capillary blood flow, or another indicator of hemodynamic performance include so-called “bi-directional” rebreathing processes, as disclosed in U.S. Pat. Nos. 6,200,271 and 6,210,342, both of which issued to Kück et al. on Mar. 13, 2001, and Apr. 3, 2001, respectively (hereinafter “the '271 patent” and “the 342 Patent”, respectively), and the data-refining methods described in International Patent Application WO 01/62148, published on Aug. 30, 2001.

In bi-directional rebreathing processes, data obtained before, during and after rebreathing are evaluated. In the data-refining methods, data obtained during conventional rebreathing processes or in bi-directional rebreathing may be evaluated and refined to eliminate unreliable data points and, thus, to provide more accurate calculations.

Typically, differential Fick techniques rely upon baseline measurements, i.e., measurements taken during “normal” respiration, of carbon dioxide elimination and the partial pressure of end-tidal carbon dioxide. Once baseline data has been gathered, a change in the effective ventilation of the individual is induced. Once the VCO₂ and PetCO₂ values become stable with the change in effective ventilation, these parameters are again measured. The difference between the baseline values and those taken during the change in the effective ventilation of the individual are used to calculate the pulmonary capillary blood flow or cardiac output of the individual.

In one example of a known differential Fick technique for inducing a change in the effective ventilation of an individual, carbon dioxide may be added to the gases that are inhaled by the individual, either directly, e.g., by the addition of carbon dioxide from a cylinder or other external source, or by causing an individual to rebreathe previously exhaled gases. An exemplary differential Fick technique that has been used, which is disclosed in Gedeon, A. et al. in 18 Med. & Biol. Eng. & Comput., 411-418 (1980) (hereinafter “Gedeon”), employs a period of increased ventilation followed immediately by a period of decreased ventilation.

When other so-called “rebreathing” processes are used, the exhaled volume of carbon dioxide may change only slightly, while the inhaled volume of carbon dioxide, which is normally negligible, may increase substantially. As a consequence, the difference between the amounts of carbon dioxide that are exhaled and inhaled during rebreathing is reduced substantially and the VCO₂ of the individual decreases to a level that is less than that which is measured during normal breathing. Rebreathing during which the VCO₂ decreases to near zero is typically referred to as “total rebreathing”. Rebreathing that causes some decrease, but not a total reduction of VCO₂, is typically referred to as “partial rebreathing”. These rebreathing processes may be used either to noninvasively estimate the CvCO₂, as in “total rebreathing”, or to obviate the need to know CvCO₂, as in “partial rebreathing”.

Rebreathing is typically conducted with a rebreathing circuit, which causes an individual to inhale a gas mixture that includes carbon dioxide. For example, the rebreathed air, which may be inhaled from a deadspace during rebreathing, includes air that was previously exhaled by the individual, i.e., carbon dioxide-rich air.

During total rebreathing, substantially all of the gas inhaled by the individual was expired during the previous breath. Thus, during total rebreathing, PetCO₂ is typically assumed to be equal, or closely related, to the partial pressure of carbon dioxide in the arterial (PaCO₂), venous (PvCO₂) and alveolar (PACO₂) blood of the individual. Total rebreathing processes are based on the assumption that neither the pulmonary capillary blood flow or cardiac output, nor the CvCO₂ of the individual, changes substantially during the rebreathing process. The partial pressure of carbon dioxide in blood may be converted to the content of carbon dioxide in blood by means of a carbon dioxide dissociation curve, where the change in the carbon dioxide content of the blood (CvCO₂−CaCO₂) is equal to the slope(s) of the carbon dioxide dissociation curve multiplied by the measured change in PetCO₂, as caused by a change in effective ventilation, such as rebreathing.

In partial rebreathing, the individual inhales a mixture of “fresh” gases and gases that were exhaled during the previous breath. Thus, the individual does not inhale a volume of carbon dioxide as large as the volume of carbon dioxide that would be inhaled during a total rebreathing process.

Conventional partial rebreathing processes typically employ a differential form of the carbon dioxide Fick equation, such as equation (4), to determine the pulmonary capillary blood flow or cardiac output of the individual. Since the carbon dioxide content of the venous blood of the individual is assumed to remain substantially the same, i.e., constant, in the periods during which measurements are obtained, knowledge of the carbon dioxide content of the venous blood of the individual is not typically required in partial rebreathing processes. Again, with a carbon dioxide dissociation curve, the measured partial pressure of end-tidal carbon dioxide can be used to determine the change in content of carbon dioxide in the blood before and during the rebreathing process. Accordingly, the following equation may be used to determine pulmonary capillary blood flow or cardiac output when partial rebreathing is conducted:

Q=VCO₂/s PetCO₂ (5)

where s is the slope of the carbon dioxide dissociation curve.

While partial rebreathing is currently the most commonly used method for causing a change in the effective ventilation of an individual, rebreathing techniques pose a number of drawbacks. In particular, setup time and labor for using rebreathing circuits can be extensive, must be adjusted to meet the needs of each individual being measured and, typically, require a fair amount of individual cooperation. When attempting to measure pulmonary capillary blood flow or cardiac output of an individual whose breathing is being aided by a mechanical ventilation machine, this setup can become extremely difficult and patient cooperation may become virtually impossible. Consequently, alternatives to rebreathing are needed to induce changes in the effective ventilation of an individual, particularly for individuals whose breathing is being aided by a mechanical ventilation machine.

B. Mechanical Ventilation

A modem mechanical ventilation machine has a wide range of modes for delivering breaths to an individual, each of which may be manipulated by one or more control parameters. As an individual's ability to breathe unassisted may vary due to the degree of ventilatory dysfunction, a mechanical ventilation machine may have to provide only some portion of the needed ventilatory support or all of the needed ventilatory support, depending upon the needs of the individual.

Ventilation is provided as volume delivered to an individual's lungs in discrete breaths. The total amount of ventilation is equal to the product of the volume of the breaths and the rate at which the breaths are delivered. The ventilation and/or the volume of the breaths may be manually or systematically set on the ventilation machine using the flow rate (i.e., the rate of breath delivery), the delivered tidal volume, and/or the inspiratory termination pressure (i.e., the pressure at which the inspiratory period of a breath will be terminated). Stated another way, a mechanical ventilation machine may be set to specify one or more of these variables depending upon the needs of the individual whose breathing is being monitored or assisted. For example, a ventilation machine may be set to a specified flow rate and a specified inspiratory termination pressure, or simply a specified tidal volume that will be delivered for each breath. Similarly, a mechanical ventilation machine may be set to deliver a volume of gas to the individual until a set inspiratory termination pressure is reached.

Mechanical ventilation machines utilize various control variables to determine when an individual's breath begins and when it ends. These control variables include time (i.e., duration of inspiration and expiration, as well as the pause time therebetween), delivered tidal volume, inspiratory pressure (both initiation pressure and termination pressure) and flow rate. In some cases, combinations of these variables are used to initiate and terminate breaths. The simplest example of a control variable is time control. In time control, the total time duration of a breath may be simply determined based upon the rate of breath delivery that is set. Thus, to vary the duration of a breath, one would simultaneously vary the flow rate set on the ventilation machine.

A common ventilatory mode is to initiate breaths according to a pre-set time sequence, e.g., one breath every six seconds, and to terminate the inspiratory period when a set inspiratory termination pressure or tidal volume has been reached. When a breath is terminated because a variable has reached a predefined threshold, the variable often is referred to as a “cycle variable”. Cycle variables may be any control variable and typically include time, inspiratory termination pressure and delivered tidal volume. By way of example, a breath may be initiated based upon a predetermined time sequence and terminated when a predetermined delivered tidal volume has been reached. Alternatively, a similarly initiated breath, or inspiration, may be terminated when a predetermined inspiratory pressure or duration has been achieved.

In addition to control variables, mechanical ventilation machines often employ limit variables to tailor delivery of breaths to an individual's needs. A limit variable is a variable which includes a threshold, i.e., a minimum and/or a maximum value, that may be attained during the course of a breath. For example, a ventilation machine may be set to provide breaths at a fixed time interval, such as ten breaths per minute, but the pressure provided to the lungs may be limited such that it never exceeds a threshold, e.g., 20 cm H₂O, during the breath. Limit variables typically include inspiratory pressure, tidal volume, flow rate and time.

Timing of breaths using a mechanical ventilation machine often is controlled using a techniques that is commonly referred to as “patient triggering”. In patient triggering, the ventilation machine detects an attempt by the individual to breathe and subsequently, if necessary, augments the breathing attempt by adding mechanical support. The individual's attempt to breathe is typically detected by the ventilation machine using inspiratory initiation pressure and/or flow rate signals. In other words, if an individual's inspiratory attempt lowers the pressure in the breathing circuit below a predetermined threshold value, the ventilation machine registers the threshold value, considers it an attempt at inspiration and provides inspired gas flow until a pre-set inspiratory termination pressure, tidal volume, or time duration has been reached. An individual may be permitted to trigger all breaths as long as a minimum number of individual-initiated breaths are observed by the ventilation machine over a given period of time. If this minimum number of breaths is not registered by the machine, one or more supplemental mandatory mechanical breaths will typically be delivered to meet a predetermined ventilation rate.

In addition to providing ventilatory support to the monitored and/or assisted individual, mechanical ventilation machines have been utilized to estimate individual respiratory parameters, such as resistance and compliance. With the addition of monitoring technologies such as flow, CO₂ and pulse oximetry, ventilation machines have been used to estimate additional hemodynamic parameters, such as measures of hemodynamic performance, e.g., pulmonary capillary blood flow, cardiac output, stroke volume, and cardiac index. In Gedeon, a version of timing control was implemented using respiratory equipment and utilized to achieve a well-controlled breath-holding procedure of short duration. In the procedure utilized by Gedeon, the length of the pause between inspiration and expiration was altered while inspiratory time and expiratory time were maintained at constant levels. Thus, a scheme akin to a very short, mandatory breath-holding period was achieved. Tidal volume and inspiration pressure also were held constant. Gedeon used both a steady-state hyperventilation scheme as well as a hyper-hypoventilation scheme to achieve the necessary two states of ventilation. The results of Gedeon's work are shown in FIG. 1.

As long as the individual is being ventilated under strict timing control, as was the primary ventilatory mode utilized in the 1970s and 1980s, the approach of Gedeon provides an effective method for calculation or estimation of pulmonary capillary blood flow and cardiac output. However, mechanical ventilation machines have evolved considerably with the addition of various modes and control parameters as previously discussed. Further, utilization of differential Fick calculations based upon deadspace rebreathing has developed considerably following the work of Gedeon (1985), Capek and Roy (1988) and others. Nonetheless, noninvasive measurements of pulmonary capillary blood flow and cardiac output continue to be based solely on the effects of rebreathing on ventilation. To the inventors' knowledge, differential Fick techniques have yet to be developed that utilize measurements taken under the wide array of ventilatory modes now available to calculate pulmonary capillary blood flow, cardiac output and other measures of hemodynamic performance.

### SUMMARY OF THE INVENTION

The present invention includes methods for inducing temporary changes in the effective ventilation of an individual by manipulating one or more respiratory control parameters. Once a change has been induced in the effective ventilation of the individual, indicators of hemodynamic performance, e.g., pulmonary capillary blood flow, cardiac output, etc., or lung perfusion, may be calculated utilizing any suitable differential Fick techniques, such as equation (4), the bi-directional rebreathing processes disclosed in the '271 and '342 patents, the data-refining methods described in WO 01/62148, or otherwise, as known in the art. The present invention further includes methods for determining lung perfusion that are simple and may be performed on individuals whose breathing is monitored and/or assisted by a mechanical ventilation machine.

In one embodiment, the present invention includes a method for estimating hemodynamic performance of an individual, for example, by estimating cardiac output and/or pulmonary capillary blood flow. According to the invention, at least two ventilatory states, which are sufficiently different from one another, are obtained or defined. Subsequently, measurements of an indicator of the carbon dioxide content of the blood of the individual are taken at each of the two ventilatory states, during a transition between the two ventilatory states, at one of the ventilatory states and during the transition between the first and second ventilatory states, or any combination of the foregoing. The measurements so obtained are applied to a differential form of the Fick equation, such as equation (4) above, a bidirectional rebreathing algorithm, or a data-refining algorithm, to estimate cardiac output and/or pulmonary capillary blood flow.

The present invention includes a number of methods for obtaining or defining the necessary two ventilatory states. Generally, a baseline, first ventilatory state is defined. Definition of the baseline ventilatory state may be effected under substantially “normal” breathing conditions. Alternatively, the baseline ventilatory state may be defined under a first set of other, selected breathing conditions. The second ventilatory state occurs under different breathing conditions than those present during the first ventilatory state, which breathing conditions are sufficiently different from one another to effect a measurable change in minute ventilation. The second ventilatory state may be induced, for example, by altering the value of a limit variable, e.g., inspiratory pressure, tidal volume, flow rate or time, from a value of the limit variable during the first ventilatory state.

In another exemplary method, a change in effective ventilation may be induced by altering the threshold value of a cycle variable from the threshold level of the cycle variable during the first ventilatory state. In a further exemplary method, a change in effective ventilation may be induced by altering the threshold triggering value of a triggering variable, such as inspiratory pressure or flow rate. In a still further method according to the present invention, a change in effective ventilation may be induced by delivering to the individual a series of at least three “sigh breaths,” which are deeper than normal breaths. Changes in effective ventilation may also comprise periods of unsteady, or “noisy,” breathing.

In each of the methods of the present invention, a measurement of the carbon dioxide content of the blood of the individual and the carbon dioxide elimination of the individual may be obtained during each of the first and second ventilatory states or during a transition between ventilatory states. The obtained measurements may be applied to a differential form of the carbon dioxide Fick equation, such as the version represented herein as equation (4), to estimate hemodynamic performance.

Optionally, more than two different ventilatory states may be induced and appropriate measurements obtained during or between each such ventilatory state to estimate hemodynamic performance by use of a variation of the Fick equation, as known in the art. For example, the measurements may be applied to a probability distribution and the relationship between the measurements evaluated to estimate cardiac output and/or pulmonary capillary blood flow.

The present invention also includes systems for effecting one or more of the inventive methods. In a system according to the present invention, different ventilatory states may be input into and/or stored by memory associated with a ventilation machine. The changes in ventilation may then be induced manually by an operator of the ventilation machine or the ventilation machine may be set to automatically induce a change in the effective ventilation of the individual. The change in effective ventilation may be induced at predetermined intervals of time, in response to changes in one or more monitored conditions (e.g., respiratory conditions, cardiac conditions, etc.) of the individual, or otherwise.

Alternatively, a conscious individual may modify his or her breathing to effect a change in effective ventilation. Such modification may be effected pursuant to predetermined instructions.

These and other objects, features and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWING

FIG. 1 graphically represents results of the work of Gedeon utilizing a version of timing control and is an illustration of how variations in pause time between inspiration and expiration may cause temporary changes in minute ventilation; and

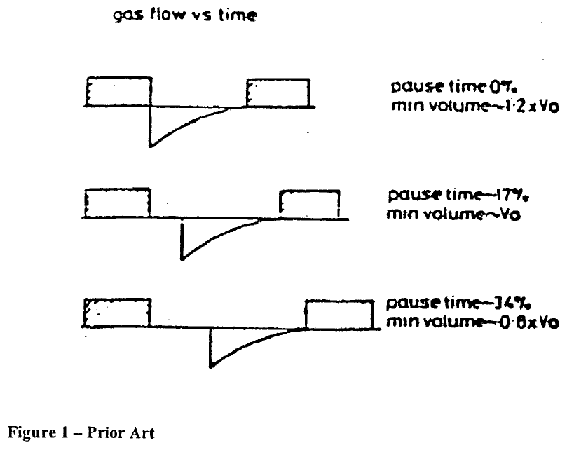

FIGS. 2 and 3 are schematic representations of systems for automatically effecting the methods of the present invention.

### DETAILED DESCRIPTION OF THE INVENTION

The present invention is directed to methods for inducing changes in the effective ventilation of an individual by manipulating one or more respiratory parameters. Measurements of carbon dioxide elimination or oxygen consumption and a respective indicator of the content of carbon dioxide or oxygen in the blood of the individual that are obtained prior to and during (or immediately following) the change in effective ventilation may be utilized to calculate one or more measures of hemodynamic performance, e.g., pulmonary capillary blood flow or cardiac output. The particular embodiments described herein are intended in all respects to be illustrative rather than restrictive. Alternative embodiments will become apparent to those skilled in the art to which the present invention pertains without departing from its scope.

The present invention relates to methods for non-invasively obtaining measurements of carbon dioxide elimination or oxygen consumption and a respective indicator of the content of carbon dioxide (e.g., partial pressure of end-tidal carbon dioxide) or oxygen in the blood of an individual. The measurements so obtained then may be applied to a differential form of the Fick equation, such as equation (4) above, a bi-directional rebreathing algorithm, or a data-refining algorithm to estimate pulmonary capillary blood flow, cardiac output, or another indicator of hemodynamic performance. For such calculations, measurements of carbon dioxide elimination or oxygen consumption and of an indicator of the respective carbon dioxide or oxygen content of an individual's blood are obtained during or at a transition between a minimum of two observable ventilatory states that are sufficiently different from one another to result in changes in the measured respiratory parameters that may be used to accurately determine the desired indicator of hemodynamic performance. The two ventilatory states may comprise a baseline ventilatory state or first ventilatory state, such as that achieved under “normal” breathing conditions, and an altered, second ventilatory state. Alternatively, neither of the ventilatory states may comprise a normal ventilatory state, so long as the first and second ventilatory states are substantially different from one another. The key to successful and accurate cardiac output and pulmonary capillary blood flow estimation is that the two ventilatory states are sufficiently different from one another to effect a measurable change in minute ventilation.

State-of-the-art ventilation machines include a wide array of modes for delivering breaths to an individual. Each mode may be manipulated by one or more control parameters, which may be manually or systematically set on the ventilation machine. A typical state-of-the-art ventilation machine permits the setting and, consequently, the manipulation of respiratory parameters including, but not limited to, flow rate (i.e., the rate of breath delivery), delivered tidal volume, inspiratory termination pressure (i.e., the pressure at which an inspiration will be terminated) and timing (i.e., duration of inspiration and expiration, as well as the pause time between the inspiratory and expiratory periods). Particular ventilation machines may permit the setting of parameters other than those discussed herein and that the methods of the present invention are equally applicable to such parameters. As such, the particular respiratory parameters discussed herein are merely intended to illustrate examples of the present invention and do not limit the scope of the present invention in any way.

Ventilation changes may be achieved by periodic or a periodic alteration of any of the ventilation machine's control variables including, but not limited to, cycle variables and/or limit variables, so long as the net effect is a change in the amount or make-up of gas that is inhaled by the individual whose breathing is being monitored or assisted. As will be more fully described below, a number of the respiratory parameters may similarly be altered consciously by an individual whose breathing is being monitored, whether or not that individual's respiration depends upon or is assisted by a ventilation machine. Such unassisted alterations of control and limit variables are also contemplated to be within the scope of the present invention.

As previously discussed, a limit variable is a variable which includes a threshold, i.e., a minimum or a maximum value, of a particular respiratory parameter that may be attained during the course of a breath. Limit variables include, without limitation, inspiratory termination pressure, delivered tidal volume, flow rate and time. By way of example, a desired ventilatory state may be induced by altering at least one limit variable. More particularly, two states of ventilation may be achieved by determining a baseline value for a limit value (i.e., creating a first ventilatory state), taking measurements of carbon dioxide elimination or oxygen consumption and a respective indicator of carbon dioxide or oxygen content in the blood under conditions of the first ventilatory state, subsequently altering the limit variable (i.e., creating the second ventilatory state) and taking a measurement of an indicator of carbon dioxide content in the blood under conditions of the second ventilatory state. The measurements taken under the two different ventilatory conditions then may be applied to a differential form of the carbon dioxide Fick equation to estimate cardiac output, pulmonary capillary blood flow, or another indicator of hemodynamic performance.

An exemplary illustration of inducing a change in effective ventilation by altering a limit variable involves altering inspiratory termination pressure. For instance, breaths may be initiated according to a predetermined timing pattern, which is maintained over both the first and second ventilatory states, but the inspiratory termination pressure of the breaths may be changed to achieve an altered, second ventilatory state. According to this exemplary method, during the first ventilatory state, in which “normal” ventilation (i.e., under baseline ventilatory conditions) occurs, inhalation is effected until a predetermined inspiratory termination pressure is reached. Measurements of carbon dioxide elimination or oxygen consumption and a respective indicator of carbon dioxide or oxygen content in the blood are then obtained at this first ventilatory state. To achieve the altered, second ventilatory state, for example, a state of hyperventilation inhalation may be effected until an inspiratory termination pressure slightly higher (e.g., approximately 10% higher) than the baseline inspiratory pressure is reached. Similarly, to achieve a state of hypoventilation, inhalation may be effected until an inspiratory termination pressure slightly less than the baseline inspiratory pressure is reached.

Measurements of carbon dioxide elimination or oxygen consumption and a respective indicator of carbon dioxide or oxygen content in the blood are then obtained at one or both of the altered states or a transitory period leading to one or both of the altered states. The values of the measurements, along with corresponding measurements obtained during the first ventilatory state, may be applied to a differential form of the Fick equation to estimate cardiac output, pulmonary capillary blood flow, or another indicator of hemodynamic performance. Alternatively, the baseline measurement could be discarded (or this step avoided altogether) and the measurements taken during both a hypoventilation state and a hyperventilation state or at a transition therebetween may be applied to a differential form of the Fick equation.

A similar method may be employed by alteration of the other limit variables, such as volume, flow rate or time. For instance, breaths may be initiated according to a predetermined timing pattern during both first and second ventilatory states, but the tidal volume of the breaths may be changed from that of a first ventilatory state to achieve an altered, second ventilatory state. Measurements of carbon dioxide elimination or oxygen consumption and a respective indicator of carbon dioxide or oxygen content in the blood taken at each of the two ventilatory states and/or during a transition therebetween may then be utilized, such as in some differential version of the Fick equation, to estimate an indicator of hemodynamic performance, such as cardiac output or pulmonary capillary blood flow.

It will be understood and appreciated by those of skill in the art that limit variables oftentimes function as safeguards, for example, to protect an individual's lungs from unduly high pressures. Therefore, it is important that variations in limit variables for the purpose of inducing a change in the effective ventilation of an individual remain relatively small, such that the safety of the individual is not affected, while still permitting two sufficiently different ventilatory states.

In another exemplary method, an altered ventilatory state may be induced by changing the ventilatory conditions for terminating inspiratory flow. More particularly, two states of ventilation may be achieved by determining a threshold value for a cycle variable creating a first ventilatory state, taking measurements of carbon dioxide elimination or oxygen consumption and a respective indicator of carbon dioxide or oxygen content in the blood under conditions of the first ventilatory state, subsequently altering the threshold value creating a second ventilatory state and taking measurements of carbon dioxide elimination or oxygen consumption and a respective indicator of carbon dioxide or oxygen content in the blood under conditions of the second ventilatory state. Alternatively, or in addition, such measurements may be obtained during a transition between the first and second ventilatory states. Such measurements may be applied to a differential form of the Fick equation to estimate cardiac output, pulmonary capillary blood flow, or another indicator of hemodynamic performance.

An exemplary illustration of altering conditions for terminating inspiratory flow by altering the threshold value for a cycle variable includes alteration of the inspiratory termination pressure. In particular, such an inspiratory termination pressure alteration may be achieved by setting a ventilation machine that communicates with an airway of a monitored individual to cycle when the inspiratory pressure of the individual's respiration reaches 20 cm H₂O. For purposes of the present example, 20 cm H₂O represents the inspiratory pressure condition of a baseline, first ventilatory state. After taking measurements of carbon dioxide elimination or oxygen consumption and a respective indicator of carbon dioxide or oxygen content in the blood at the first ventilatory state, an altered, second ventilatory state, during which the individual hyperventilates, may be achieved by setting the inspiratory pressure threshold to a value higher than that of the baseline state, e.g., 22 cm H₂O. Similarly, an altered, second ventilatory state, during which the individual hypoventilates, may be achieved by setting the inspiratory pressure threshold to a value lower than that of the baseline state, e.g., 18 cm H₂O. 1511 As with the previous example, measurements of carbon dioxide elimination or oxygen consumption and a respective indicator of carbon dioxide or oxygen content in the blood may be taken at one or both of the hyperventilation and hypoventilation states. Alternatively, or in addition, such measurements may be obtained at a transition to one or both of the hyperventilation and hypoventilation states. Measurements obtained during two or more different ventilatory states or transitions therebetween may then be used in a differential form of the Fick equation to estimate an indicator of hemodynamic performance, such as cardiac output or pulmonary capillary blood flow.

Alternative embodiments of methods that incorporate teachings of the present invention include altering the thresholds of other cycle variables, such as tidal volume or time, to induce a change in the effective ventilation of an individual.

As will be understood and appreciated by those of skill in the art, inspiration may be effected in the above examples by either a ventilation machine or consciously by the individual whose breathing is being monitored or assisted. If the individual were consciously effecting their own inspiration, he or she may be prompted by way of computer display or the like when the appropriate limit to pressure, volume, flow rate, or time had been achieved. For example, a computer display may prompt the individual to breath more deeply (volume control), or faster (time control), for a pre-set number of breaths to induce a desired change in effective ventilation from a first ventilatory state to a second ventilatory state.

In a further embodiment of a method of inducing a change in effective ventilation according to the present invention, timing of an individual's ventilation may be altered. As discussed above, Gedeon utilized a timing alteration technique wherein the length of the pause between the inspiratory period and expiratory period was altered while inspiration and expiration time remained the same. Two sufficiently different ventilatory states also may be achieved by, instead, altering the durations of one or both of the inspiratory and expiratory periods while causing the pause time therebetween to remain substantially constant. Measurements of carbon dioxide elimination or oxygen consumption and a respective indicator of carbon dioxide or oxygen content in the blood may then be obtained at the two different ventilatory states or during a transition therebetween and utilized in a differential form of the Fick equation to estimate hemodynamic performance.

As will be understood and appreciated by those of skill in the art, alterations in the inspiratory and expiratory periods may be effected in the above method by either a ventilation machine or consciously by the individual whose breathing is being monitored or assisted. If the individual were consciously altering their own periods of inspiration and expiration, he/she may be prompted by way of computer display or the like when the appropriate inspiratory or expiratory duration is achieved.

As previously discussed, timing of breaths using a ventilation machine often is controlled using “patient triggering”, wherein the ventilation machine detects an attempt by the monitored individual to breathe and subsequently, if necessary, augments the breathing attempt with mechanical support. An individual's attempt to breathe may be detected by the ventilation machine using inspiration initiation pressure and/or flow rate signals. As such, an altered ventilatory state for purposes of calculating a measure of hemodynamic performance by use of a differential form of the carbon dioxide Fick equation may be achieved by altering a trigger level, or predetermined threshold for a particular respiratory parameter, at which breathing attempts are detected by the ventilation machine, such as an inspiratory pressure or flow rate trigger level.

For instance, the amount of inspiratory pressure or inspiratory flow needed to signal an individual's attempt to breathe may be altered to create another level of ventilation and, thus, a different ventilatory state. By way of example, if the predetermined inspiratory pressure threshold for baseline breathing attempts is initially set at −2 cm H₂O and the ventilation machine is not responsible for the individual's breathing, a brief period of hypoventilation may be created by altering the trigger threshold to a lower value, such as −2.5 cm H₂O. Conversely, if the ventilation machine is at least partially responsible for the individual's breathing, a brief period of hyperventilation may be created by altering the trigger threshold to a higher value, such as −1.5 cm H₂O. Again, measurements of carbon dioxide elimination or oxygen consumption and a respective indicator of carbon dioxide or oxygen content in the blood of the monitored individual may be taken prior to and during (or immediately following) one of the altered ventilatory states or, alternatively, during both of the altered ventilatory states. The values so obtained may then be applied to a differential form of the Fick equation to estimate an indicator of hemodynamic performance.

In patient triggering, an individual is typically permitted to trigger all breaths, as long as a minimum number of individual-initiated breaths are observed and registered by the ventilation machine over a given period of time. If the minimum number of breaths is not registered by the ventilation machine, supplemental mandatory mechanical breaths are delivered to meet a predetermined ventilation rate. Accordingly, another exemplary embodiment of a method for inducing a change in effective ventilation in accordance with the present invention includes changing the number of mandatory breaths that must be delivered in a given time frame. For instance, if ten breaths per minute are delivered by the ventilation machine in a first ventilatory state, a change in the effective ventilation of an individual and, thus, a different ventilation state may be achieved by changing the mandatory number of breaths that are to be delivered by the ventilation machine to either a lower number or a higher number, for example, to nine or eleven breaths per minute.

Measurements of carbon dioxide elimination or oxygen consumption and a respective indicator of carbon dioxide or oxygen content in the blood may then be taken and applied to a differential form of the Fick equation, as previously described. As will be understood and appreciated by those of skill in the art, alteration of the mandatory number of delivered breaths may be problematic from a safety standpoint if the number of breaths delivered falls below about eight breaths per minute.

Some ventilation machines include a mode wherein so-called “sigh breaths” are delivered. Sigh breaths are periodic breaths that are of considerably larger volume than “normal” breaths. Accordingly, a further embodiment of a method for inducing a change in effective ventilation according to the present invention includes delivery of a series of a minimum or three “sigh breaths”. Again, measurements of carbon dioxide elimination or oxygen consumption and a respective indicator of carbon dioxide or oxygen content in the blood may be taken both before and upon delivery of (or immediately following) the “sigh breath” series. The measurements so obtained may then be applied to a differential form of the Fick equation to calculate an indicator of hemodynamic performance, such as cardiac output or pulmonary capillary blood flow.

As will be understood and appreciated by those of skill in the art, an altered ventilation state using “sigh breaths” may also be achieved in individuals whose breathing is not assisted or monitored by a ventilation machine. If the individual is capable of consciously effecting his or her own inspiration, he or she may be prompted, for example by way of computer display, another individual, written instructions, or the like, to inhale and exhale more deeply than normal for a particular series of breaths. Accordingly, this method of inducing a change in effective ventilation may be utilized with individuals whether or not their breathing is being assisted or mechanically monitored.

Further, the present invention is not limited to measurements taken at only two ventilatory states or transitions therebetween. Estimation of cardiac output and pulmonary capillary blood flow may be achieved by creating any number of different states of ventilation and obtaining measurements of carbon dioxide elimination or oxygen consumption and a respective indicator of carbon dioxide or oxygen content in the blood at each ventilatory state or a transition between ventilatory states. Such measurements then may be applied to probabilistic distributions for estimating hemodynamic performance. Using this approach, it may be possible to reduce the impact a particular change in ventilation has on an individual.

The use of probabilistic distributions to vary the control variables and/or limit variables of ventilation machines recently has been discussed in the medical literature in the context of recruiting alveoli and improving gas exchange. See, e.g., Mutch, W. A. et al., Biologically Variable Ventilation Increases Arterial Oxygenation Over That Seen With Positive End-Expiratory Pressure Alone in a Porcine Model of Acute Respiratory Distress Syndrome, 28 (7) Crit. Care Med., 2457-64 (July 2000) (hereinafter “Mutch 18(7)”); Mutch, W. A. et al., Biologically Variable or Naturally Noisy Mechanical Ventilation Recruits Atelectatic Lung, 162 (1) Am J. Respir. Crit. Care Med., 319-23 (July 2000) (hereinafter “Mutch 162(1)”); Arold et al., Variable Tidal Volume Ventilation Improves Lung Mechanics and Gas Exchange in a Rodent Model of Acute Lung Injury, 165 Am J. Respir Crit Care Med., 366-371 (2002); Boker et al., Improved Arterial Oxygenation with Biologically Variable or Fractal Ventilation Using Low Tidal Volumes in a Porcine Model of Acute Respiratory Distress Syndrome, 165, Am J Respir Crit Care Med. 456-462 (2002). To the inventors' knowledge, applying probabilistic distributions to the measurement of indicators of hemodynamic performance, such as cardiac output or pulmonary capillary blood flow, has not been employed prior to the present invention.

Biologically variable ventilation (BVV) has been found in recent studies to improve arterial oxygenation over conventional ventilatory control modes. (See, Arold; Boker; Mutch 18(7); Mutch 162(1); Mutch, W.A et al., Biologically Variable Ventilation Prevents Deterioration of Gas Exchange During Prolonged Anesthesia, 84(2) Br. J. Anaesth., 197-203 (February 2000)). It is also within the scope of the present invention to vary respiratory parameters, such as the delivered tidal volume, flow rate and timing, using probability distribution functions (PDFs) derived directly or indirectly from subjects not under the control of a ventilation machine. The resulting variability (i.e., standard deviation or other such measure) is typically sufficiently large to provide sufficient changes in ventilation so that estimates of an indicator of hemodynamic performance may be made. Some modes of ventilation, such as pressure support ventilation, appear to provide the variability in these parameters in at least some settings.

For example, Hotchkiss et al. in Oscillations and Noise: Inherent Instability of Pressure Support Ventilation?, 165(1) Am. J. Respir. Crit. Care Med., 47-53 (January 2002), found that the pressure support ventilation in the setting of airflow obstruction can be accompanied by marked variations in delivered tidal volume and end-expiratory alveolar pressure, even when subject effort is unvarying. Thus, varying respiratory limits and parameters in a probabilistic sense may provide similar monitoring capabilities but in a less intrusive manner and, therefore, is also within the scope of the present invention.

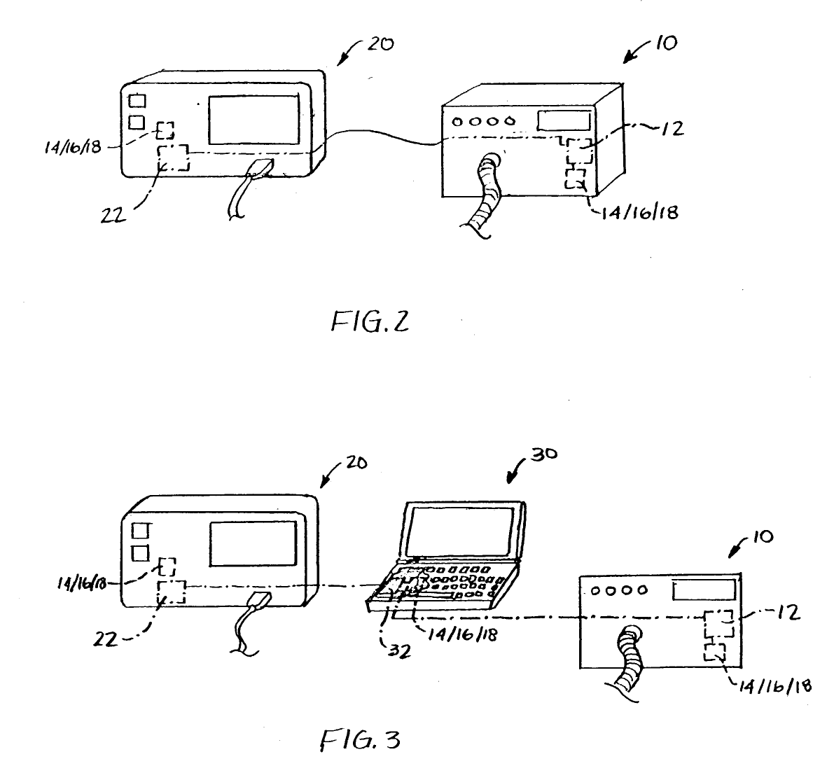

Any combination of the preceding methods for inducing changes in effective ventilation may be used to change the effective target alveolar ventilation, as measured by an apparatus which is capable of noninvasively measuring hemodynamic performance, such as the NICO® and CO₂SMO PLUS!® monitors, both of which are offered commercially by Respironics, Inc. of Pittsburgh, Pa. One or more respiratory parameters measured with such an apparatus may be manually input into a processor (either internal or external) that controls operation of the ventilation machine or, as depicted in FIG. 2, transmitted, as known in the art and substantially in real-time, from a processor 22 of the hemodynamic performance-measuring apparatus 20 to the processor 12 that controls operation of the ventilation machine 10. Processor 22 of hemodynamic performance-measuring apparatus 20, processor 12 of ventilation machine 10, or an intermediate processor 32 of an independent control unit 30, as shown in FIG. 3, may operate under control of one or more algorithms or programs which effect one or more methods of the present invention.

The one or more algorithms or programs may be designed to select and cause the ventilation machine to effect the safest change in effective ventilation for the individual being monitored. The one or more algorithms or programs may be embodied as known in the art, such as in the form of software stored on a memory device 14, firmware 16, or programmed hardware 18 that is in communication with the executing processor 12, 22, 32. Of course, other suitable arrangements of systems incorporating teachings of the present invention are also within the scope of the present invention.

In conclusion, the present invention includes methods for inducing changes in the effective ventilation of an individual by manipulating one or more respiratory parameters, measurements derived as a result of such changes facilitating calculation of an indicator of hemodynamic performance using a differential form of the Fick equation. The present invention has been described in relation to particular embodiments which are intended in all respects to be illustrative rather than restrictive. Alternative embodiments will become apparent to those skilled in the art to which the present invention pertains without departing from its scope.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims.

What is claimed is:

1. A method of estimating hemodynamic performance of an individual, comprising:
causing an individual to effect a first breath at a first set of ventilatory conditions including a ventilation parameter, wherein the ventilation parameter comprises inspiratory pressure, flow rate, tidal volume, inspiratory duration, expiratory duration, or a combination thereof;
altering the ventilation parameter to define a second set of ventilatory conditions; and
causing such an individual to effect a second breath at the second set of ventilatory conditions.

2. The method of claim 1, wherein the altering is manually performed.

3. The method of claim 1, further comprising:
obtaining first measurements of (a) carbon dioxide elimination or oxygen consumption and (b) an indicator of carbon dioxide content or oxygen content in blood of the individual at the first set of ventilatory conditions;
obtaining second measurements of (a) the carbon dioxide elimination or oxygen consumption and (b) the indicator of carbon dioxide content or oxygen content of blood of such an individual at the second set of ventilatory conditions; and
applying the first and the second measurements to a differential Fick equation.

4. The method of claim 3, wherein obtaining the first and the second measurements of the indicator of carbon dioxide content comprises obtaining a first measurement and a second measurement of partial pressure of end-tidal carbon dioxide.

5. The method of claim 3, further comprising inputting the first set of ventilatory conditions into a mechanical ventilation machine.

6. The method of claim 5, wherein the altering is performed automatically by the mechanical ventilation machine.

7. The method of claim 1, further comprising:
obtaining first measurements of (a) carbon dioxide elimination or oxygen consumption and (b) an indicator of carbon dioxide content or oxygen content in blood of the individual at the first set of ventilatory conditions or at the second set of ventilatory conditions;
obtaining second measurements of (a) the carbon dioxide elimination or oxygen consumption and (b) the indicator of carbon dioxide content or oxygen content of blood of such an individual at a transition between the first set of ventilatory conditions and the second set of ventilatory conditions; and
applying the first and the second measurements to a differential Fick equation.

8. A method for estimating hemodynamic performance of an individual using a differential Fick technique, comprising:
determining a first value indicative of carbon dioxide content in the blood of a individual at a first ventilatory state, the first ventilatory state including a ventilation parameter, wherein the ventilation parameter comprises inspiratory pressure, flow rate, tidal volume, inspiratory duration, expiratory duration, or a combination thereof;
altering the ventilation parameter to define a second ventilatory state;
determining a second value indicative of carbon dioxide content in the blood of such an individual at the second ventilatory state; and
estimating hemodynamic performance by applying the first value and the second value to a differential Fick equation.

9. The method of claim 8, wherein determining the first and the second values indicative of carbon dioxide content include obtaining a first measurement and a second measurement of a partial pressure of end-tidal carbon dioxide.

10. The method of claim 8, wherein the altering is manually performed.

11. The method of claim 8, wherein the first ventilatory state is input into a mechanical ventilation machine.

12. The method of claim 11, wherein the altering is performed automatically by the mechanical ventilation machine.

13. A method for determining at least one of cardiac output and pulmonary capillary blood flow of an individual, comprising:
(1) defining a baseline ventilatory state including a baseline value of at least one of (a) a limit variable other than time, (b) a cycle variable other than time, and (c) a triggering variable for triggering an inspiratory phase of a breathing cycle;
(2) obtaining a first measurement of (a) carbon dioxide elimination or oxygen consumption and (b) an indicator of carbon dioxide content or oxygen content of blood of an individual at the baseline ventilatory state;
(3) defining an altered ventilatory state including an altered value of the at least one of the limit variable, the cycle variable, and the triggering variable;
(4) obtaining a second measurement of (a) the carbon dioxide elimination or oxygen consumption and (b) the indicator of carbon dioxide content or oxygen content of blood of such an individual at the altered ventilatory state; and
(5) applying the first measurement and the second measurement to a differential Fick equation.

14. The method of claim 13, further comprising:
effecting an inspiratory period at the baseline ventilatory state prior to obtaining the first measurements; and
effecting an inspiratory period at the altered ventilatory state prior to obtaining the second measurements.

15. The method of claim 13, wherein the obtaining the first measurement and the second measurement of the indicator of the carbon dioxide content comprises obtaining a first measurement and a second measurement of partial pressure of end-tidal carbon dioxide.

16. The method of claim 13, further comprising inputting the baseline ventilatory state and the altered ventilatory state into a mechanical ventilation machine.

17. The method of claim 16, wherein defining the altered ventilatory state is performed automatically by the mechanical ventilation machine.

18. The method of claim 13, wherein defining an altered ventilatory state is manually performed.

19. A method for estimating at least one of cardiac output and pulmonary capillary blood flow of an individual utilizing a mechanical ventilation machine, comprising:
obtaining a baseline value of at least one of (a) a limit variable other than time, (b) a cycle variable other than time, and (c) a triggering variable for triggering an inspiratory phase of a breathing cycle;
storing the baseline value in the mechanical ventilation machine;
obtaining an altered value of the at least one of the limit variable, the cycle variable, and the triggering variable; and
storing the altered value in the mechanical ventilation machine.

20. The method of claim 19, further comprising:
determining a first measurement of (a) carbon dioxide elimination or oxygen consumption and (b) an indicator of carbon dioxide content or oxygen content of blood of such an individual at the baseline value;
determining a second measurement of (a) the carbon dioxide elimination or oxygen consumption and (b) the indicator of carbon dioxide content or oxygen content of blood of such an individual at the altered value; and
applying the first measurement and the second measurement to a differential Fick equation.

21. The method of claim 19, further comprising:
determining a first measurement of (a) carbon dioxide elimination or oxygen consumption and (b) an indicator of carbon dioxide content or oxygen content of blood of such an individual at the baseline value or at the altered value;
determining a second measurement of (a) the carbon dioxide elimination or oxygen consumption and (b) the indicator of carbon dioxide content or oxygen content of blood of such an individual at a transition between the baseline value and the altered value; and
applying the first measurement and the second measurement to a differential Fick equation.

22. The method of claim 19, wherein determining the first measurement and the second measurement of the indicator of the carbon dioxide content comprises obtaining a first measurement and a second measurement of a partial pressure of end-tidal carbon dioxide.

23. The method of claim 19, further comprising causing the individual to effect an inspiratory period and an expiratory period at the baseline value and at the altered value of the limit variable.

24. A method for inducing a measurable change in ventilation of an individual utilizing a ventilation machine, comprising:
defining a first ventilatory state including a ventilation parameter, the ventilation parameter comprising an inspiratory pressure, flow rate, tidal volume, inspiratory duration, expiratory duration, or combination thereof;
altering the at least one ventilation parameter to define a second ventilatory state; and
effecting an inspiratory period and an expiratory period at the first and the second ventilatory states.

25. The method of claim 24, wherein the altering is manually performed.

26. The method of claim 24, further comprising:
obtaining a first measurement of (a) carbon dioxide elimination or oxygen consumption and (b) an indicator of at least one of carbon dioxide content or oxygen content of blood of such an individual at the first ventilatory state;
obtaining a second measurement of (a) the carbon dioxide elimination or oxygen consumption and (b) the indicator of carbon dioxide content or oxygen content of blood of such an individual at the second ventilatory state; and
applying the first measurement and the second measurement to a differential Fick equation.

27. The method of claim 26, wherein the obtaining the first and the second measurement of the indicator of the carbon dioxide content comprises obtaining a first measurement and a second measurement of a partial pressure of end-tidal carbon dioxide.

28. The method of claim 24, further comprising:
obtaining a first measurement of (a) carbon dioxide elimination or oxygen consumption and (b) an indicator of at least one of carbon dioxide content or oxygen content of blood of such an individual at the first ventilatory state or at the second ventilatory state;
obtaining a second measurement of (a) the carbon dioxide elimination or oxygen consumption and (b) the indicator of carbon dioxide content or oxygen content of blood of such an individual at a transition between the first ventilatory state and the second ventilatory state; and
applying the first measurement and the second measurement to a differential Fick equation.

29. The method of claim 24, further comprising inputting the first ventilatory state into the ventilation machine.

30. The method of claim 29, wherein the altering is performed automatically by the ventilation machine.

31. A method of estimating hemodynamic performance of an individual, comprising:
determining a first value indicative of carbon dioxide content of blood of the individual at a first ventilatory state;
determining a second value indicative of carbon dioxide content of blood of the individual at a second ventilatory state, wherein the second ventilatory state comprises a series of at least three sigh breaths; and
applying the first value and the second value to a differential Fick equation.

32. The method of claim 31, wherein the determining the first and second values indicative of carbon dioxide content comprise obtaining first and second values of a partial pressure of end-tidal carbon dioxide.

33. A method of estimating hemodynamic performance of an individual utilizing differential Fick techniques, comprising:
obtaining a plurality of measurements of (a) carbon dioxide elimination or oxygen consumption and (b) an indicator of carbon dioxide content or oxygen content of blood of such an individual at a plurality of ventilatory states; and
applying the plurality of measurements to a probabilistic distribution.

34. A method for estimating hemodynamic performance of an individual using a differential Fick technique, comprising:
providing a first ventilatory state for an individual, wherein the first ventilatory state includes a ventilation parameter, wherein the ventilation parameter comprises inspiratory pressure, flow rate, tidal volume, inspiratory duration, expiratory duration, or a combination thereof;
altering the ventilation parameter to define a second ventilatory state;
determining a first value indicative of carbon dioxide content in the blood of a individual at the first ventilatory state or at the second ventilatory state;
determining a second value indicative of carbon dioxide content in the blood of such an individual at a transition between the first ventilatory state and the second ventilatory state; and
estimating hemodynamic performance by applying the first value and the second value to a differential Fick equation.

35. The method of claim 34, wherein determining the first and the second values indicative of carbon dioxide content include obtaining a first measurement and a second measurement of a partial pressure of end-tidal carbon dioxide.

36. The method of claim 34, wherein the altering is manually performed by the individual.
